(19) United States
(12) Patent Application Publication
(10) Pub. No.: US 2024/0177869 A1
(43) Pub. Date: May 30, 2024

(54) METHOD AND SYSTEM FOR GENERATING A PLURALITY OF ANTIBODY SEQUENCES OF A TARGET FROM ONE OR MORE FRAMEWORK REGIONS

(71) Applicant: Innoplexus AG, Eschborn (DE)

(72) Inventors: Sudhanshu Kumar, Bokaro (IN); Joel Joseph, Palayi (IN); Ansh Gupta, Menhdawal (IN)

(73) Assignee: Innoplexus AG, Eschborn (DE)

(21) Appl. No.: 18/060,187

(22) Filed: Nov. 30, 2022

(51) Int. Cl. G16H 70/40; G06N 3/0442

(52) U.S. Cl. CPC G16H 70/40; G06N 3/0442

(57) ABSTRACT

A method and system for generating a plurality of antibody sequences of a target from one or more framework regions based on at least one model. The model is trained on a training dataset of high binding affinity to generate the complementarity determining regions (CDR) from the received one or more framework regions (FR). The generated complementarity determining regions (CDR) from the each of the one or more framework regions (FR) are combined with the associated one or more framework regions to generate one or more regions of the target. The generated one or more regions comprises each of the received one or more framework regions (FR) and corresponding each of the generated complementarity determining regions (CDR). The generated one or more regions are concatenated to generate the plurality of antibody sequences of the target. The generated plurality of antibody sequences of the target has high binding affinity.

### FIELD OF TECHNOLOGY

Certain embodiments of the disclosure relate to generating a plurality of antibody sequences of a target from one or more framework regions. More specifically, certain embodiments of the disclosure relate to a method and system for generating a plurality of antibody sequences of a target from one or more framework regions.

### BACKGROUND

Designing and generating biomolecules with a known function has increasingly become one of the major goals of biotechnology and biomedicine over the past few decades. Development of cheaper techniques to synthesize and sequence DNA has led to this. However, the total possible number of Protein Sequences is so large that Deep Mutational Scanning and even very large libraries barely scratch the surface of possibilities.

Antibody-Antigen binding affinity maturation is an important challenge in the field of drug development. Antigens and Antibodies are both proteins i.e., Antibody sequences. An antigen is usually a disease-causing entity and so if a suitable antibody is introduced which binds well with the antigen at the correct paratope region, then it means that the functionality of the antigen is neutralized. Currently, the major ways of designing antibodies are semi laboratory-based techniques dependent on applying certain intuitions over lab data. The computational methods are limited to frequency-based methods. Therefore, coming up with an increasingly in-silico drug development method would accelerate drug discovery and development.

In-silico Antibody Affinity maturation i.e., generating antibody sequences which have high binding affinity is a very challenging problem in drug discovery. Typically, the whole sequence is generated at a time and then the sequences that fit the biological rules are selected. Although this technique provides the sequences however, there is no way of ascertaining that the sequences generated have high binding affinity.

Further limitations and disadvantages of conventional and traditional approaches will become apparent to one of skill in the art, through comparison of such systems with some aspects of the present disclosure as set forth in the remainder of the present application with reference to the drawings.

### SUMMARY

The aspects of the disclosed embodiments are directed to generate a plurality of antibody sequences for a target with high binding affinity.

One aspect of the disclosed embodiments is directed to generating a plurality of complementarity determining regions (CDR) for each of the received one or more framework regions (FR)

Another aspect of the disclosed embodiments is directed to generating one or more regions of the plurality of the antibody sequences from the received one or more framework regions of the target

Further aspects of the disclosed embodiments are directed to concatenate the generated one or more regions to generate the plurality of antibody sequences for the target.

Yet another aspect of the disclosed embodiments is directed to train at least one model to generate the plurality complementarity determining regions (CDR) from one or more framework regions (FR).

Further, another aspect of the disclosed embodiments is directed to pre-process a plurality of known antibody sequences of the target to generate a training dataset for training the at least one model.

Furthermore, another aspect of the disclosed embodiments is directed to train the at least one model to generate the plurality of antibody sequences with high binding affinity.

A method is disclosed for generating a plurality of antibody sequences of a target from one or more framework regions, substantially as shown in and/or described in connection with at least one of the figures, as set forth more completely in the claims.

These and other advantages, aspects and novel features of the present disclosure, as well as details of an illustrated embodiment thereof, will be more fully understood from the following description and drawings.

### BRIEF DESCRIPTION OF SEVERAL VIEWS OF THE DRAWINGS

FIG. 1 is a block diagram that illustrates an exemplary system for generating a plurality of antibody sequences of a target from one or more framework regions, in accordance with an exemplary embodiment of the disclosure.

FIG. 2 depicts visual representation of an antibody sequence, in accordance with an exemplary embodiment of the disclosure.

FIG. 3 is a block diagram that illustrates an exemplary system for training the at least one model on a training dataset to generate the complementarity determining regions (CDR), in accordance with an exemplary embodiment of the disclosure.

FIG. 4 is a block diagram that illustrates an exemplary system for generating the complementarity determining regions (CDR) and combining the one or more framework regions (FR) to generate the plurality of antibody sequences, in accordance with an exemplary embodiment of the disclosure.

FIG. 5 depict flowcharts illustrating exemplary operations for generating the plurality of antibody sequences of the target from one or more framework regions, in accordance with various exemplary embodiments of the disclosure.

FIG. 6 depict flowcharts illustrating exemplary operations for pre-processing a received plurality of antibody sequence of the target to generate the training dataset, in accordance with various exemplary embodiments of the disclosure.

### DETAILED DESCRIPTION OF THE DISCLOSURE

Certain embodiments of the disclosure relate to generating a plurality of antibody sequences of a target from one or more framework regions. In the context of the current invention, a plurality of complementarity determining regions (CDR) is generated for each of the received one or more framework regions (FR), and the generated complementarity determining regions (CDR) is combined with each of the received one or more framework regions (FR) to generate one or more regions. The one or more regions are subsequently concatenated to generate the plurality of antibody.

In accordance with various embodiments of the disclosure, a method is provided for generating a plurality of antibody sequences of a target from one or more framework regions. The method comprises receiving, by one or more processors, one or more framework regions of one or more regions of an antibody sequence of the target, wherein the one or more regions of an antibody sequence comprises of one or more framework regions and one or more complementarity determining regions (CDR). The method further comprises generating, by one or more processors, a plurality complementarity determining regions (CDR) for each of the one or more framework regions (FR), wherein the complementarity determining regions (CDR) is generated based on at least one model. Further, the method comprises combining, by one and more processors, the each of the received one or more framework regions (FR) and each of the generated complementarity determining regions (CDR) corresponding to each of the received one or more framework regions to generate one or more regions. Furthermore, the method comprises concatenating, by one or more processors, the one or more regions to generate the plurality of antibody sequences of the target.

In accordance with an embodiment, the at least one model is trained on a training dataset to generate the complementarity determining regions (CDR) from the each of the one or more framework regions (FR).

In accordance with an embodiment, the method comprises pre-processing a plurality of known antibody sequences of the target to generate the training dataset.

In accordance with various embodiments of the disclosure, pre-processing the plurality of antibody sequence of the target comprises processing, by one or more processors, the plurality of known antibody sequence of the target to identify one or more regions in the plurality of known antibody sequences, wherein each region of the one or more regions comprises of one or more known framework regions (FR) and one or more known complementarity determining regions (CDR). The method further comprises padding, by one or more processors, the one or more known framework regions (FR) and one or more known complementarity determining regions (CDR) of the one or more regions with #to equalize lengths of the one or more regions of the plurality of known antibody sequences. The method further comprises concatenating, by one or more processors, the padded one or more known framework regions (FR) and the one or more known complementarity determining regions (CDR). Furthermore, the method comprises inserting, by one or more processors, spaces between each character of the concatenated plurality of known antibody sequences. The method further comprises removing, by one or more processors, unidentified antibody from the concatenated plurality of known antibody sequence to generate the training dataset.

In accordance with an embodiment, the at least one model comprises one of Autoregressive Convolutional Neural Network, Long Short-Term Memory (LSTM) networks, Markov model, and GPT-2 model.

In accordance with an embodiment, each of the sequences of the training dataset is converted into one-hot encoding to provide the one-hot encoded training dataset to the autoregressive CNN model for generating the complementarity determining regions (CDR) from the each of the received one or more framework regions (FR).

In accordance with an embodiment, the Long Short-Term Memory (LSTM) networks comprises embedding layer to learn vocabulary of the training dataset to generate the complementarity determining regions (CDR) from the each of the received one or more framework regions (FR).

In accordance with an embodiment, the Markov model extracts frequency and other parameters from the training dataset to generate the complementarity determining regions (CDR) from each of the received one or more framework regions (FR).

In accordance with an embodiment, the GPT-2 model implements one or more techniques to generate the complementarity determining regions (CDR) from the each of the received one or more framework regions (FR).

In accordance with an embodiment, the plurality of known antibody sequence of the target and the generated plurality of antibody sequences of the target has high binding affinity.

In accordance with another aspect of the disclosure, a system for generating a plurality of antibody sequences of a target from one or more framework regions. The system comprises at least one server communicable coupled with at least one database. The server comprising one or more processors configured to receive one or more framework regions of one or more regions of an antibody sequence of the target, wherein the one or more regions of an antibody sequence comprises of one or more framework regions and one or more complementarity determining regions (CDR), generate, based on at least one model, a plurality complementarity determining regions (CDR) for each of the received one or more framework regions (FR), combine each of the received one or more framework regions (FR) and each of the generated complementarity determining regions (CDR) corresponding to each of the received one or more framework regions to generate one or more regions, and concatenate the one or more regions to generate the plurality of antibody sequences of the target.

In accordance with an embodiment, the at least one model is trained on a training dataset to generate the complementarity determining regions (CDR) from the each of the one or more framework regions (FR).

In accordance with an embodiment, the at least one server is configured to pre-process a plurality of known antibody sequence of the target to generate the training dataset.

In accordance with another aspect of the disclosure, wherein the at least one server is configured to process the plurality of known antibody sequence of the target to identify one or more regions in the plurality of known antibody sequence, wherein each region of the one or more regions comprises of one or more framework regions (FR) and one or more complementarity determining regions (CDR), padding the one or more known framework regions (FR) and one or more known complementarity determining regions (CDR) of the one or more regions with #to equalize lengths of the one or more regions of the plurality of known antibody sequences, concatenating the padded one or more known framework regions (FR) and the one or more known complementarity determining regions (CDR), inserting spaces between each character of the concatenated plurality of known antibody sequence and removing unidentified antibodys from the concatenated plurality of known antibody sequence to generate the training dataset.

In accordance with an embodiment, the at least one model comprises one of Autoregressive Convolutional Neural Network, Long Short-Term Memory (LSTM) networks, Markov model, and GPT-2 model. The inventor expects skilled artisans to employ one or more model and its variations as appropriate. The models mentioned herein should not be considered limiting, and the inventors intend for the present invention to be practiced otherwise than specifically described herein.

In accordance with an embodiment, each of the sequences of the training dataset is converted into one-hot encoding to provide the one-hot encoded training dataset to the autoregressive CNN model for generating the complementarity determining regions (CDR) from the each of the received one or more framework regions (FR).

In accordance with an embodiment, the Long Short-Term Memory (LSTM) networks comprises embedding layer to learn vocabulary of the training dataset to generate the complementarity determining regions (CDR) from the each of the received one or more framework regions (FR).

In accordance with an embodiment, the Markov model extracts frequency and other parameters from the training dataset to generate the complementarity determining regions (CDR) from the each of the received one or more framework regions (FR).

In accordance with an embodiment, the GPT-2 model implements one or more techniques to generate the complementarity determining regions (CDR) from the each of the received one or more framework regions (FR).

In accordance with an embodiment, the plurality of known antibody sequence of the target and the generated plurality of antibody sequences of the target has high binding affinity.

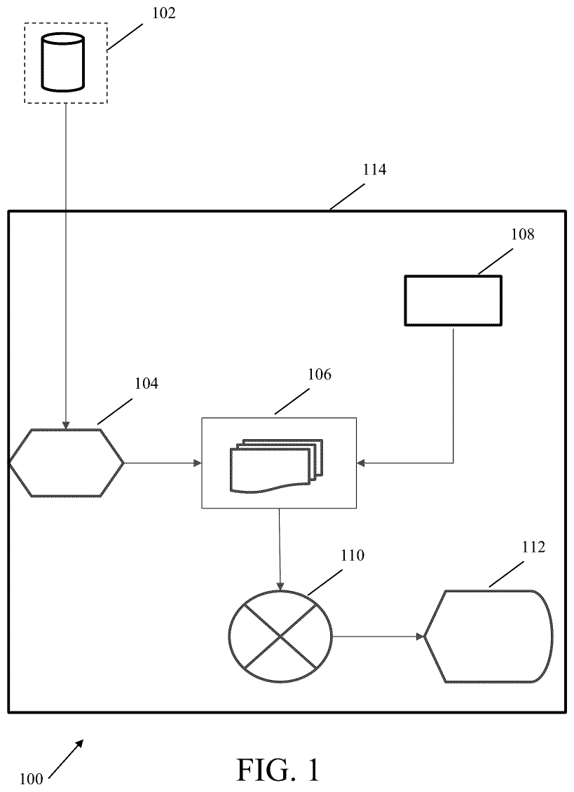

FIG. 1 is a block diagram that illustrates an exemplary system for generating a plurality of antibody sequences. Referring to FIG. 1, a system 100 includes at least one server 114 and at least one database arrangement 102. The at least one server 114 comprises of a pre-processing module 104, a training and generation module 106, an input module 108, a combination and concatenation module 110, and output module 112. The at least one server 114 and the database arrangement 102 is communicably coupled via the communication network (not shown).

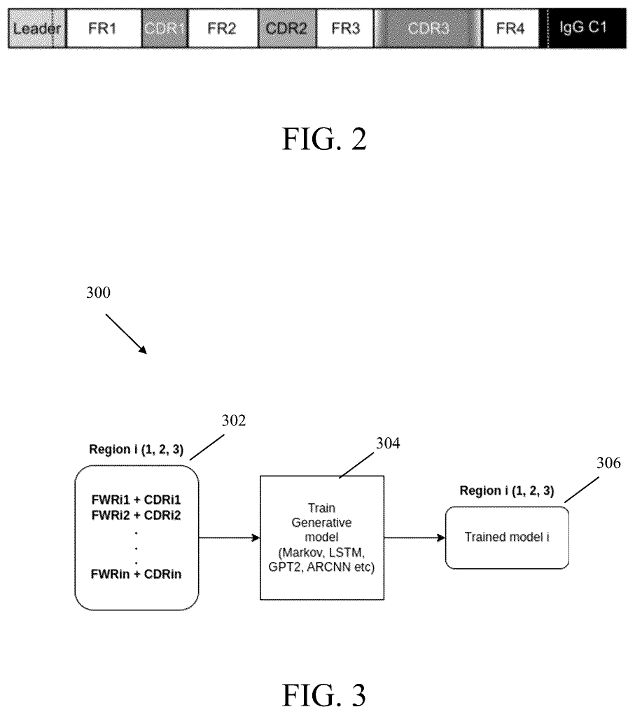

The system 100 is configured to receive a plurality of antibody sequences. The nucleotide sequences in FASTA format are converted into the antibody sequences using IMGT tool or any other tool suitable for said purpose. The antibody sequence usually consists of alphabetical characters where FR (framework regions) are usually fixed (low mutation) and CDR (complementarity determining regions)/HV (hyper variable) regions vary (high mutation). These sequences are divided into regions, 4 FR and 3 CDR as provided in FIG. 2. There are 20 antibody that occur naturally in nature, which can be represented by a three or single letter code as follows: Alanine (Ala, A); Arginine (Arg, R); Asparagine (Asn, N); Aspartic acid (Asp, D); Cysteine (Cys, C); Glutamic acid (Glu, E); Glutamine (Gln, Q); Glycine (Gly, G); Histidine (His, H); Isoleucine (Ile, I); Leucine (Leu, L); Lysine (Lys, K); Methionine (Met, M); Phenylalanine (Phe, F); Proline (Pro, P); Serine (Ser, S); Threonine (Thr, T); Tryptophan (Trp, W); Tyrosine (Tyr, Y); Valine (Val, V). In an embodiment, the received antibody sequences are divided into 3 regions. Each region contains FR and consecutive CDR regions. The plurality of regions associated with the received plurality of antibody sequences are then used for compiling a training dataset. One or more models are trained using the training dataset to generate CDR based on the prefix FR of antibody. Referring to FIG. 2, it has been observed that FR4 is generally constant and has minimum to no mutation, whereas FR 1, 2, and 3 are also mostly constant but sometimes we observe few mutations. CDR on the other hand have very high mutations. The at least one server 114 is configured to receive only the constant part of FR regions as prefix and generate mutating part of FR as well as whole CDR corresponding to it. For example, let's say FR1 is of length 17 (position 1-17) and CDR1 is of length 8 (position 18-25) characters. But FR position 16 and 17 are identified to be mutating to some extent. Then in that case, we will pass FR1 with positions 1-15 as prefix and generate positions from 16-25.

The at least one server 114 further comprises a memory, a storage device, an input/output (I/O) device, a user interface, and a wireless transceiver. The at least one database 102 is external or remote but communicatively coupled to the at least one server 114 via a communication network.

In some embodiment of the disclosure, the pre-processing module 104, the training and generation module 106, an input module 108, a combination and concatenation module 110, and output module 112 are integrated with other processors and modules to form an integrated system. In some embodiments of the disclosure the one or more processors of the at least one server 114 may be integrated in any order and other combination modules to form an integrated system. In some embodiments of the disclosure, as shown, the pre-processing module 104, the training and generation module 106, an input module 108, a combination and concatenation module 110, and output module 112 may be distinct from each other. Other separation and/or combination of the various processing engines and entities of the exemplary system 100 illustrated in FIG. 1 may be done without departing from the spirit and scope of the various embodiments of the disclosure.

The at least one database 102 is configured to store the plurality of known antibody sequences of the target. The database may be capable of providing mass storage to the at least one server 114. In some embodiments, the database contains a computer-readable medium, such as a hard disk device, an optical disk device, or a tape device, a flash memory or other similar solid state memory device, or an array of devices, including devices in a storage area network or other configurations. A computer program product may be tangibly embodied in an information carrier. The information carrier may be a computer-readable or machine-readable medium, such as database. The computer program product may also contain instructions that, when executed, perform one or more methods, such as those described in the disclosure. In an embodiment, the received plurality of antibody sequence has high binding affinity value. Further, the received plurality of antibody sequence of the target should correspond to one particular target. Moreover, each of the received plurality of antibody sequence of the target corresponds to one particular paratope.

The pre-processing module 104 comprises of suitable libraries, logic, and/or code that may be operable to pre-process the received plurality of antibody sequence of the target in conjunction with the one or more processors. More specifically, the pre-processing module, in conjunction with the one or more processors, enables the at least one server 114 to generate a training dataset suitable of training the training and generation module 106. In an embodiment, the pre-processing module 104 receives the plurality of known antibody sequence of the target from the database arrangement 102. The plurality of known antibody sequence are pre-existing sequences known to have high binding affinity. In an embodiment, the pre-processing module 104 is configured to process the plurality of known antibody sequence of the target to identify one or more regions in the plurality of known antibody sequence. The one or more regions in the plurality of known antibody sequence is identified by the pre-processing module 104 using one or more standard bio-informatic algorithms. Each of the one or more regions comprises one or more known framework regions (FR) and one or more known complementarity determining regions (CDR). In an embodiment, the pre-processing module 104 is configured to pad the one or more known framework regions (FR) and one or more known complementarity determining regions (CDR) of the one or more regions with #to equalize lengths of the one or more regions of the plurality of known antibody sequences. Thereafter, the pre-processing module 104 is configured to concatenate the padded one or more known framework regions (FR) and the one or more known complementarity determining regions (CDR), insert spaces between each character of the concatenated plurality of known antibody sequences, and remove unidentified antibodys from the concatenated plurality of known antibody sequence to generate the training dataset. In an embodiment, the pre-processing module 104 is configured to insert spaces between the characters to convert the characters into information capable of being interpreted contextually by the training and generation module 106. In an embodiment, inserting spaces between the characters helps the at least model consider each amino acid to be treated as independent token and thus enables generation of the next amino acid in the CDR (sometime FR). In an embodiment, the spaced characters are identified as antibody by the training and generation module 106 to train the at least one model. Further, the unidentified antibodies are removed by the pre-processing module 104 from the concatenated plurality of known antibody sequence to generate the training dataset. In an embodiment, the removal of the unidentified antibodies helps in removing the noise. In an embodiment, the pre-processing module 104 is configured to split the received plurality of known antibody sequences into a training dataset and test dataset. The test dataset is further processed to drop the duplicate antibody sequences.

The training and generation module 106 comprises suitable libraries, logic, and/or code operable to implement the training and generation function in conjunction with the one or more processors. More specifically, the training and generation module, in conjunction with the one or more processors, enables the at least one server 114 to generate the plurality of antibody sequences of a target from one or more framework regions. In an embodiment, the training and generation module 106 is configured to train one of at least one module that comprises one of a Autoregressive Convolutional Neural Network, Long Short-Term Memory (LSTM) networks, Markov model, and GPT-2 (Generative Pre-trained Transformer-2) model. In an embodiment, the training and generation module 106 is configured to receive the training dataset from the pre-processing module 104 to train the at least one model. In an embodiment, the training and generation module 106 is configured to generate the plurality complementarity determining regions (CDR) for each of the received one or more framework regions (FR) based on at least one model.

The training and generation module 106 comprises suitable libraries, logic, and/or code operable to train the at least one model 300 in reference to FIG. 3. In an embodiment, the training and generation module 106 is configured to receive the training dataset from the pre-processing module 104 to train the at least one model 300. In an embodiment, the training dataset comprises one or more regions of the known antibody sequences of the target. The one or more regions 302 comprises one or more known framework regions (FR) and one or more known complementarity determining regions (CDR) associated with the one or more known framework regions (FR). The one or more regions 302 is provided to the at least one generative model 304. In an embodiment, the at least one generative model 304 comprises one of Autoregressive Convolutional Neural Network, the Long Short-Term Memory (LSTM) networks, the Markov model and the GPT-2 model. In an embodiment, the at least one generative model 304 is configured to receive the one or more regions associated with the training dataset to identify sequential relationship between the one or more known framework regions (FR) and the one or more known complementarity determining regions (CDR) associated with the one or more framework regions (FR) of the target. In an embodiment, the model 306 is operable to implement one of the at least one model to generate the complementarity determining regions (CDR) from the received one or more framework regions (FR) of one region of the one or more regions. In an example, the training dataset for a target comprises of 3 regions (Region 1,2,3), wherein each region of (Region 1,2,3) comprises of pairs of FR and CDR. The at least one generative model 304 is operable to generate 3 model (Trained model 1, trained model 2, trained model 3) 306 for each of the 3 regions. Thus, during generation upon receiving one or more framework regions from the input module, the training and generation model 106 will operate the model 306 to generate the corresponding one or more complementarity determining regions (CDR). In an example, the one or more region 302 comprises a FR1 of length 17 (position 1-17) and CDR1 is of length 8 (position 18-25) characters. The at least one generative model 304 306 identifies that FR position 16 and 17 to be mutating to some extent. In an example, in the FR is not mutating then the at least one generative model considers the whole FR (1-17) as a prefix for generating the CDR. The at least one generative model 304 learns to use one of the models 306 to receive FR1 with positions 1-15 as prefix and generate CDR positions for 16-25.

The at least one generative model 304 of the training and generation module 106 comprises of Autoregressive Convolutional Neural Network, the Long Short-Term Memory (LSTM) networks, the Markov model and the GPT-2 model. In an embodiment, the at least one generative model 304 comprises suitable libraries, logic, and/or code that are operable to build the model 306. In an embodiment, each of the one or more regions of the training dataset 302 is converted into one-hot encoding to provide the one-hot encoded training dataset to the autoregressive CNN model for building the model 306 to generate the complementarity determining regions (CDR) from the each of the one or more framework regions (FR). In an embodiment, the Long Short-Term Memory (LSTM) networks comprises embedding layer to learn vocabulary of the training dataset 302 to generate the complementarity determining regions (CDR) from the each of the one or more framework regions (FR) as per the model 306. In an embodiment, the Markov model extracts frequency and other parameters from the training dataset 302 to build the model 306 to generate the complementarity determining regions (CDR) from the each of the one or more framework regions (FR). In an embodiment, the GPT-2 model implements one or more techniques to train the training dataset 302 keeping to build the model 306.

In an embodiment, the Markov Chain model a stochastic model, i.e., the model is based on random probability distribution. Markov Chain models the future state i.e., in case of text generation, the next word, based on the previous state (i.e., previous word or sequence). The model is memory-less —the prediction depends only on the current state of the variable (it forgets the past states; it is independent of preceding states). On the other hand, it's simple, fast to execute and light on memory.

In an embodiment, using Markov Chain model for text generation requires the following steps:
a. Load the dataset and preprocess text.
b. Extract from text the sequences of length n (current state) and the next words (future state).
c. Build the transition matrix with the probability values of state transitions.

In order to build a transition matrix, the Markov chain model processes the entire text and counts all transitions from a particular sequence (ngram) to the next word. The values are then stored in a matrix, where rows correspond to the particular sequence and columns to the particular token (next word). The values represent the number of occurrences of each token after the particular sequence. Since the transition matrix should contain probabilities, not counts, in the end the occurrences are recalculated into probabilities. The matrix is saved in scipy.sparse format to limit the space it takes up in the memory. Thereafter the next word is predicted based on the probability distribution for state transition.

In an embodiment, Long Short-Term Memory neural networks used in classification, translation and text generation. The Long Short-Term Memory neural networks generalize across sequences rather than learn individual patterns, which makes the model a suitable tool for modeling sequential data. In order to generate text, they learn how to predict the next word based on the input sequence. The step-by-step approach for text Generation with LSTM:
a) Load the dataset and preprocess text. Extract sequences of length n (X, input vector) and the next words (y, label).
b) Build DataGenerator that returns batches of data.
c) Define the LSTM model and train it.
d) Predict the next word based on the sequence.

In an embodiment, Open AI GPT-2 is used which is a transformer-based, autoregressive language model that shows competitive performance on multiple language tasks, especially (long form) text generation. GPT-2 is trained on 40 GB of high-quality content using the simple task of predicting the next word. The model does it by using attention. It allows the model to focus on the words that are relevant to predicting the next word. Hugging Face Transformers library provides everything you need to train/fine-tune/use transformers models. The model is implemented as follows:
a) Load Tokenizer and Data Collator
b) Load data and create a Dataset object
c) Load the Model
d) Load and setup the Trainer and Training Arguments
e) Fine-tune the model
f) Generate text with the Pipeline

In an embodiment, an autoregressive CNN based sequence generation model uses 1 dimensional CNNs on the one hot encoding matrix of the sequence. During training, its input is the masked training sequence and expected output is the unmasked sequence. It uses reconstruction loss. During inference, input is the prefix added with masks and output is the prefix added with the generated fragment of the sequence.

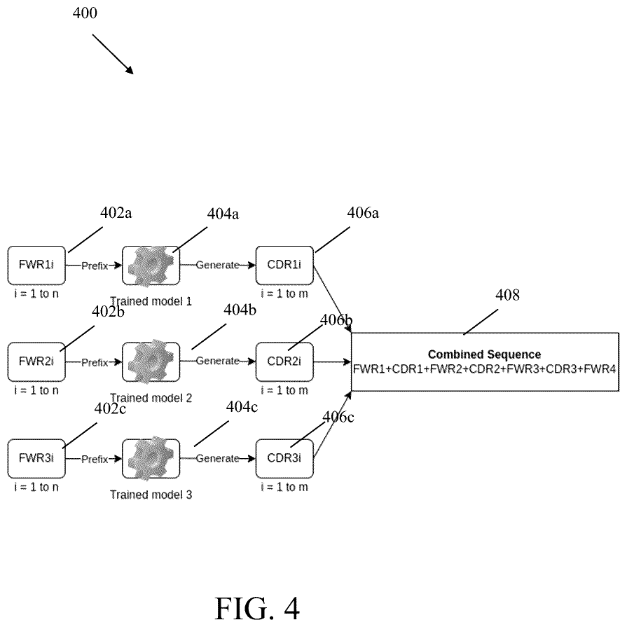

The input module 108 is operable to provide the one or more framework regions of the target to the training and generation module 106. The input module 108 comprises of suitable libraries, logic, and/or code that may be operable to receive one or more framework regions of one or more regions of an antibody sequence of the target to implement the model 306 of the training and generation module 106 to generate the corresponding complementarity determining regions (CDR) for each of the received one or more framework regions (FR). Referring to FIG. 4, the input module 108 is operable to invoke the training and generation module 106 to generate a plurality complementarity determining regions (CDR) for each of the received one or more framework regions (FR) based on the model 306. In an embodiment, the input module provides the one or more framework regions (FR) to the training and generation module 106. The training and generation model 106 receives an input framework regions (FR) 402a and invokes at least one model 404a from the at least one generative model 304 to generate a complementarity determining regions (CDR) 406a for the input framework regions (FR) 402a. The input module provides one or more input framework regions (FR) 402b and 402c, to invoke at least one model 404b and 404c to generate complementarity determining regions (CDR) 406b and 406c respectively. In an embodiment, the input module 108 is operable to provide 3 input framework regions (FR) to generate corresponding complementarity determining regions (CDR) for each of the 3 input framework regions.

The combination and concatenation module 110 comprising suitable libraries, logic, and/or code operable to implement combination and concatenation function in conjunction with the one or more processors. The combination and concatenation module 110 is operable to receive the generated plurality of complementarity determining regions (CDR) for each of the received one or more framework regions (FR) and the one or more framework regions (FR). The combination and concatenation module 110 is operable to combine the each of the received one or more framework regions (FR) and each of the generated complementarity determining regions (CDR) corresponding to each of the received one or more framework regions to generate one or more regions. In an embodiment, the combination and concatenation module 110 is operable to concatenate the one or more regions to generate the plurality of antibody sequences of the target. Referring to FIG. 4, the combined sequence 408 is operable to combine and concatenate the received one or more framework regions (FR) 402a, 402b, 402c and the generated complementarity determining regions (CDR) for each of the received one or more framework regions (FR) 406a, 406b, 406c to generate a plurality of antibody sequences 408. In an embodiment, the combination and concatenation module 110 is operable to receive 3 pairs of received framework regions (FR) and generated complementarity determining regions (CDR) for every iteration of generating the antibody sequence. In an embodiment, the received plurality of antibody sequences of the target for preparing the training dataset 302 has a high binding affinity. The generative models 304 are trained on the training dataset 302 to generate the complementarity determining regions (CDR) for each of the received one or more framework regions (FR) 406a, 406b, 406c to generate plurality of antibody sequences 408 with high binding affinity.

The output module 112 comprise suitable logic, circuitry, and interfaces configured to present the results i.e., generated the plurality of antibody sequences of the target. The results are presented in form of an audible, visual, tactile, or other output to the user, such as a researcher, a scientist, a principal investigator, data manager, and a health authority, associated with the at least one server 114. As such, the user interface may include, for example, a display, one or more switches, buttons, or keys (e.g., a keyboard or other function buttons), a mouse, and/or other input/output mechanisms. In an example embodiment, the user interface may include a plurality of lights, a display, a speaker, a microphone, and/or the like. In some embodiments, the user interface may also provide interface mechanisms that are generated on the display for facilitating user interaction. Thus, for example, the user interface may be configured to provide interface consoles, web pages, web portals, drop down menus, buttons, and/or the like, and components thereof to facilitate user interaction.

The communication network may be any kind of network, or a combination of various networks, and it is shown illustrating exemplary communication that may occur between the at least one database 102 and the at least one server 114. For example, the communication network may comprise one or more of a cable television network, the Internet, a satellite communication network, or a group of interconnected networks (for example, Wide Area Networks or WANs), such as the World Wide Web. Accordingly, other exemplary modes may comprise uni-directional or bi-directional distribution, such as packet-radio, and satellite networks.

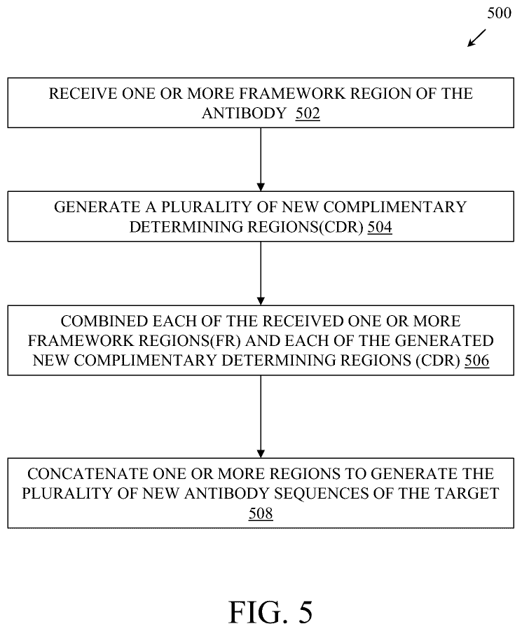

FIG. 5 flowcharts illustrates exemplary operations for generating a plurality of antibody sequences of a target from one or more framework regions. Flowcharts 500 described in conjunction with FIG. 1

At step 502, one or more framework regions of the antibody sequence corresponding to the target is received. In accordance with an embodiment, the received one or more framework region of one or more regions of antibody sequence of the target is provided to the training and generation module 106.

At step 504, a plurality of complementarity determining regions (CDR) are generated for each of the received one or more framework regions (FR), wherein the complementarity determining regions (CDR) are generated based on at least one model.

At step 506, the each of the received one or more framework regions (FR) are combined with each of the generated complementarity determining regions (CDR) corresponding to each of the received one or more framework regions to generate one or more regions.

At step 508, one or more regions are concatenated to generate the plurality of antibody sequences of the target.

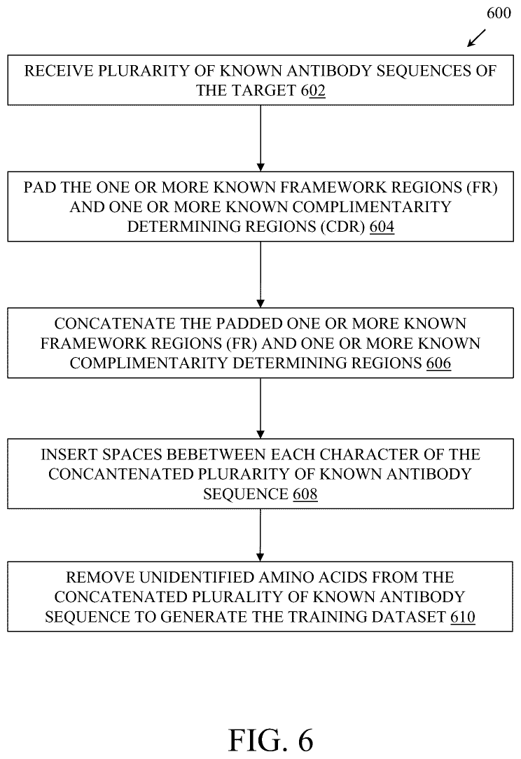

FIG. 6 flowcharts illustrating exemplary operations to pre-process a received plurality of antibody sequence of the target to generate the training dataset. Flowcharts 600 described in conjunction with FIG. 1

At step 602, a plurality of known antibody sequences of the target are received to identify one or more regions in the plurality of known antibody sequence. In accordance with an embodiment, each region of the one or more regions comprises of one or more known framework regions (FR) and one or more known complementarity determining regions (CDR).

At step 604, one or more known framework regions (FR) and one or more known complementarity determining regions (CDR) are padded of the one or more regions with #to equalize lengths of the one or more regions of the plurality of known antibody sequences.

At step 606, the padded one or more known framework regions (FR) and the one or more known complementarity determining regions (CDR) are concatenated.

At step 608, the spaces between each character on the concatenated plurality of known antibody sequence are inserted to identify the antibody sequences.

At step 610, the unidentified antibodies from the concatenated plurality of known antibody sequence are removed to generate the training dataset.

Certain embodiments of the present invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the present invention to be practiced otherwise than specifically described herein. Accordingly, this invention includes all modifications and equivalents of the subject matter recited in the claims appended hereto as permitted by applicable law. Moreover, any combination of the above-described embodiments in all possible variations thereof is encompassed by the invention unless otherwise indicated herein or otherwise clearly contradicted by context.

Groupings of alternative embodiments, elements, or steps of the present invention are not to be construed as limitations. Each group member may be referred to and claimed individually or in any combination with other group members disclosed herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

As utilized herein, the term “exemplary” means serving as a non-limiting example, instance, or illustration. As utilized herein, the terms “e.g.,” and “for example” set off lists of one or more non-limiting examples, instances, or illustrations. As utilized herein, circuitry is “operable” to perform a function whenever the circuitry comprises the necessary hardware and/or code (if any is necessary) to perform the function, regardless of whether performance of the function is disabled, or not enabled, by some user-configurable setting.

The terminology used herein is for the purpose of describing particular embodiments only and is not intended to be limiting of embodiments of the disclosure. As used herein, the singular forms “a”, “an” and “the” are intended to include the plural forms as well, unless the context clearly indicates otherwise. It will be further understood that the terms “comprises”, “comprising”, “includes” and/or “including”, when used herein, specify the presence of stated features, integers, steps, operations, elements, and/or components, but do not preclude the presence or addition of one or more other features, integers, steps, operations, elements, components, and/or groups thereof.

Further, many embodiments are described in terms of sequences of actions to be performed by, for example, elements of a computing device. It will be recognized that various actions described herein can be performed by specific circuits (e.g., application specific integrated circuits (ASICs)), by program instructions being executed by one or more processors, or by a combination of both. Additionally, these sequences of actions described herein can be considered to be embodied entirely within any non-transitory form of computer readable storage medium having stored therein a corresponding set of computer instructions that upon execution would cause an associated processor to perform the functionality described herein. Thus, the various aspects of the disclosure may be embodied in a number of different forms, all of which have been contemplated to be within the scope of the claimed subject matter. In addition, for each of the embodiments described herein, the corresponding form of any such embodiments may be described herein as, for example, “logic configured to” perform the described action.

Another embodiment of the disclosure may provide a non-transitory machine and/or computer-readable storage and/or media, having stored thereon, a machine code and/or a computer program having at least one code section executable by a machine and/or a computer, thereby causing the machine and/or computer to perform the steps as described herein for determining combination drug and use in pancreatic cancer treatment.

The present disclosure may also be embedded in a computer program product, which comprises all the features enabling the implementation of the methods described herein, and which when loaded in a computer system is able to carry out these methods. Computer program in the present context means any expression, in any language, code or notation, either statically or dynamically defined, of a set of instructions intended to cause a system having an information processing capability to perform a particular function either directly or after either or both of the following: a) conversion to another language, code or notation; b) reproduction in a different material form.

Further, those of skill in the art will appreciate that the various illustrative logical blocks, modules, circuits, algorithms, and/or steps described in connection with the embodiments disclosed herein may be implemented as electronic hardware, computer software, firmware, or combinations thereof. To clearly illustrate this interchangeability of hardware and software, various illustrative components, blocks, modules, circuits, and steps have been described above generally in terms of their functionality. Whether such functionality is implemented as hardware or software depends upon the particular application and design constraints imposed on the overall system. Skilled artisans may implement the described functionality in varying ways for each particular application, but such implementation decisions should not be interpreted as causing a departure from the scope of the present disclosure.

The methods, sequences and/or algorithms described in connection with the embodiments disclosed herein may be embodied directly in firmware, hardware, in a software module executed by a processor, or in a combination thereof. A software module may reside in RAM memory, flash memory, ROM memory, EPROM memory, EEPROM memory, registers, hard disk, physical and/or virtual disk, a removable disk, a CD-ROM, virtualized system or device such as a virtual server or container, or any other form of storage medium known in the art. An exemplary storage medium is communicatively coupled to the processor (including logic/code executing in the processor) such that the processor can read information from, and write information to, the storage medium. In the alternative, the storage medium may be integral to the processor.

While the present disclosure has been described with reference to certain embodiments, it will be noted understood by, for example, those skilled in the art that various changes and modifications could be made and equivalents may be substituted without departing from the scope of the present disclosure as defined, for example, in the appended claims. In addition, many modifications may be made to adapt a particular situation or material to the teachings of the present disclosure without departing from its scope. The functions, steps and/or actions of the method claims in accordance with the embodiments of the disclosure described herein need not be performed in any particular order. Furthermore, although elements of the disclosure may be described or claimed in the singular, the plural is contemplated unless limitation to the singular is explicitly stated. Therefore, it is intended that the present disclosure is not limited to the particular embodiment disclosed, but that the present disclosure will include all embodiments falling within the scope of the appended claims.

What is claimed is:

1. A method for generating a plurality of antibody sequences of a target from one or more framework regions, comprising:
receiving one or more framework regions of one or more regions of an antibody sequence of the target, wherein the one or more regions of an antibody sequence comprises of one or more framework regions and one or more complementarity determining regions (CDR),
generating a plurality of complementarity determining regions (CDR) for each of the received one or more framework regions (FR), wherein the plurality of complementarity determining regions (CDR) is generated based on at least one model, and
combining the each of the received one or more framework regions (FR) and each of the generated complementarity determining regions (CDR) corresponding to each of the received one or more framework regions to generate one or more regions, and
concatenating the one or more regions to generate the plurality of antibody sequences of the target.

2. The method of claim 1, wherein the method comprises pre-processing a plurality of known antibody sequences of the target to generate a training dataset.

3. The method of claim 2, wherein pre-processing comprises of
processing the plurality of known antibody sequences of the target to identify one or more regions in the plurality of known antibody sequences, wherein each region of the one or more regions comprises of one or more known framework regions (FR) and one or more known complementarity determining regions (CDR),
padding the one or more known framework regions (FR) and one or more known complementarity determining regions (CDR) of the one or more regions with #to equalize lengths of the one or more regions of the plurality of known antibody sequences,
concatenating the padded one or more known framework regions (FR) and the one or more known complementarity determining regions (CDR),
inserting spaces between each character of the concatenated plurality of known antibody sequence to identify the antibodies, and
removing unidentified antibodies from the concatenated plurality of known antibody sequence to generate the training dataset.

4. The method as claimed in claim 1, wherein the at least one model comprises one of Autoregressive Convolutional Neural Network, Long Short-Term Memory (LSTM) networks, Markov model, and GPT-2 model.

5. The method as claimed in claim 2, wherein each of the sequences of the training dataset is converted into one-hot encoding to provide the one-hot encoded training dataset to the autoregressive CNN model for generating the complementarity determining regions (CDR) from the each of the received one or more framework regions (FR).

6. The method as claimed in claim 4, wherein the Long Short-Term Memory (LSTM) networks comprises embedding layer to learn vocabulary of the training dataset to generate the complementarity determining regions (CDR) from the each of the received one or more framework regions (FR).

7. The method as claimed in claim 4, wherein the Markov model extracts frequency and other parameters from the training dataset to generate the complementarity determining regions (CDR) from the each of the received one or more framework regions (FR).

8. The method as claimed in claim 4, wherein the GPT-2 model implements one or more techniques to generate the complementarity determining regions (CDR) from the each of the received one or more framework regions (FR).

9. The method as claimed in claim 1, the plurality of known antibody sequence of the target and the generated plurality of antibody sequences of the target has high binding affinity.

10. A system for generating a plurality of antibody sequences of a target from one or more framework regions, comprising:
at least one server communicable coupled with at least one database, comprising of one or more processors configured to
receive one or more framework regions of one or more regions of an antibody sequence of the target, wherein the one or more regions of an antibody sequence comprises of one or more framework regions and one or more complementarity determining regions (CDR),
generate a plurality of complementarity determining regions (CDR) for each of the received one or more framework regions (FR), wherein the complementarity determining regions (CDR) is generated based on at least one model, and
combine the each of the received one or more framework regions (FR) and each of the generated complementarity determining regions (CDR) corresponding to each of the received one or more framework regions to generate one or more regions, and
concatenate the one or more regions to generate the plurality of antibody sequences of the target.

11. The system as claimed in claim 10, wherein the at least one server is configured to pre-process a plurality of known antibody sequence of the target to generate a training dataset.

12. The system as claimed in claim 11, wherein the at least one server is configured to
process the plurality of known antibody sequences of the target to identify one or more regions in the plurality of known antibody sequence, wherein each region of the one or more regions comprises of one or more known framework regions (FR) and one or more known complementarity determining regions (CDR),
pad the one or more known framework regions (FR) and one or more known complementarity determining regions (CDR) of the one or more regions with #to equalize lengths of the one or more regions of the received plurality of known antibody sequences,
concatenate the padded one or more known framework regions (FR) and the one or more known complementarity determining regions (CDR),
insert spaces between each character of the concatenated plurality of known antibody sequence to identify the antibodies, and
remove unidentified antibodies from the concatenated plurality of known antibody sequence to generate the training dataset.

13. The system as claimed in claim 10, wherein the at least one model comprises one of Autoregressive Convolutional Neural Network, Long Short-Term Memory (LSTM) networks, Markov model, and GPT-2 model.

14. The system as claimed in claim 11, wherein each of the sequences of the training dataset is converted into one-hot encoding to provide the one-hot encoded training dataset to the autoregressive CNN model for generating the complementarity determining regions (CDR) from the each of the received one or more framework regions (FR).

15. The system as claimed in claim 13, wherein the Long Short-Term Memory (LSTM) networks comprises embedding layer to learn vocabulary of the training dataset to generate the complementarity determining regions (CDR) from the each of the received one or more framework regions (FR).

16. The system as claimed in claim 13, the Markov model extracts frequency and other parameters from the training dataset to generate the complementarity determining regions (CDR) from the each of the received one or more framework regions (FR).

17. The system as claimed in claim 13, wherein the GPT-2 model implements one or more techniques to generate the complementarity determining regions (CDR) from the each of the received one or more framework regions (FR).

18. The system as claimed in claim 10, the plurality of known antibody sequence of the target and the generated plurality of antibody sequences of the target has high binding affinity.
